(19) United States
(12) Patent Application Publication
(10) Pub. No.: US 2007/0249576 A1
(43) Pub. Date: Oct. 25, 2007

(54) Cefdinir Oral Suspension

(76) Inventor: Chetan P. Pujara, Gurnee, IL (US)

Correspondence Address: Steven F. Weinstock;Abbott Laboratories, D-377/AP6A, 100 Abbott Park Road, Abbott Park, IL, 60064-6008, US

(21) Appl. No.: 11/766,137

(22) Filed: Jun. 21, 2007

Related U.S. Application Data
(63) Continuation of application No. 10/731,932, filed on Dec. 10, 2003, now abandoned.

(51) Int. Cl. A61K 31/546; C07D 501/58

(52) U.S. Cl. 514/207; 540/228

(57) ABSTRACT

The present invention discloses a novel powder for oral suspension of cefdinir. Also disclosed are methods of preparing the suspension and methods of treatment using the suspension.

### TECHNICAL FIELD

The present invention discloses a novel oral suspension of cefdinir. Also disclosed are methods of preparing the suspension and methods of treatment using the suspension.

### BACKGROUND OF THE INVENTION

Omnicef® for oral suspension contains the active ingredient cefdinir, an extended-spectrum, antibiotic in the cephalosporin family. Chemically, cefdinir is 7-[2-(2-aminothiazol-4-yl) -2-hydroxyiminoacetamido]-3-vinyl-3-cephem-4-carboxylic acid (syn isomer). Cefdinir is active against a wide spectrum of bacteria, including Staphylococcus aureus, Streptococcus pneumoniae, Streptococcus pyogenes, Hemophilus influenzae, Moraxella catarrhalis, E. coli, Klebsiella pneumoniae, and Proteus mirabilis.

Given the large pediatric population that uses antibiotic suspension products, compliance is a critical issue. The recommended dosage of treatment with a pediatric patient is typically based on the weight of the patient. A 1999 study showed that young patient age was associated with a lower compliance in taking oral antibiotic suspensions (Clinical Therapeutics, 1999, 21, 1193-1201). One of the factors cited as contributing to the low compliance rate in the youngest children was technical difficulty in administration of the suspensions (e.g., spillage). In a study of acute otitis media, 53% of children took less than half the prescribed medication (J. Pediatr, 1975; 87:137-141).

Omnicef® for oral suspension is indicated for the treatment of pediatric patients with acute bacterial otitis media and pharyngitis/tonsillitis. Omnicef® for oral suspension is delivered to pharmacies as a 4% (4.2% actual) cefdinir by weight powder. Upon reconstitution with water, Omnicef® is administered orally and is currently formulated as a 125 mg/5 mL suspension. In younger pediatrics, a typical dosing of Omnicef® suspension requires two 5 mL aliquots of the suspension. Administering two consecutive 5 mL aliquots can result in the loss of substantial material due to spillage. Furthermore, high concentration suspensions can show physical stability issues.

A high concentration, stable formulation that allows for the administration of a single aliquot would prove beneficial.

### SUMMARY OF THE INVENTION

In its principle embodiment the present invention provides a powder for oral suspension of cefdinir comprising greater than 4.2% by weight of cefdinir.

### DETAILED DESCRIPTION OF THE INVENTION

In its principle embodiment the present invention provides a powder for oral suspension of cefdinir comprising greater than 4.2% by weight of cefdinir.

In another embodiment the present invention provides a powder for oral suspension of cefdinir comprising between about 6% to about 10% by weight of cefdinir.

In another embodiment the present invention provides a powder for oral suspension of cefdinir comprising at least 8.4% by weight of cefdinir.

In another embodiment the present invention provides a powder for oral suspension of cefdinir comprising

(a) at least 8.4% by weight cefdinir;

(b) a diluent; and

(c) a buffering agent.

In another embodiment the present invention provides a powder for oral suspension of cefdinir comprising:

(a) about 8.4% by weight cefdinir;

(b) about 89.2% by weight diluent;

(c) about 0.26% by weight buffering agent;

(d) about 0.16% by weight preservative;

(e) about 0.33% by weight viscosity enhancer;

(f) about 1.31% by weight flavoring agent;

(g) about 0.07% glidant; and

(h) about 0.35% lubricant.

In another embodiment the present invention provides an powder for oral suspension of cefdinir comprising:

(a) about 8.36% by weight cefdinir;

(b) about 89.16% by weight sucrose;

(c) about 0.16% by weight citric acid;

(d) about 0.10% by weight sodium citrate;

(e) about 0.16% by weight sodium benzoate;

(f) about 0.16% by weight xantham gum;

(g) about 0.16% by weight guar gum;

(h) about 1.31% by weight flavoring agent;

(i) about 0.06% colloidal silicon dioxide; and

(j) about 0.35% magnesium stearate.

The present invention also teaches a method of treating acute bacterial otitis media, pharyngitis and tonsillitis with a oral suspension of cefdinir wherein said suspension is made by reconstituting a powder comprising greater than 4.2% by weight of cefdinir.

A further embodiment of the present invention teaches a method of treating acute bacterial otitis media, pharyngitis and tonsillitis with a oral suspension of cefdinir wherein said suspension is made by reconstituting a powder comprising at least 8.4% cefdinir.

All publications, issued patents, and patent applications cited herein are hereby incorporated by reference in their entirety. In the case of inconsistencies, the present disclosure, including definitions, will prevail.

As used herein, the singular forms “a”, “an”, and “the” include plural reference unless the context clearly dictates otherwise.

As used in the present specification the following terms have the meanings indicated:

The term “buffering agent,” as used herein, refers to an agent or a mixture of agents that can maintain the original acidity or basicity of a composition. Representative buffering agents include, but are not limited to, citric acid, sodium citrate, sodium phosphate, potassium citrate, and mixtures thereof. A preferred buffering agent of the present invention is a mixture of citric acid and sodium citrate.

The term “diluent,” as used herein, refers to an agent or mixture of agents that when added to a formulation makes that formulation thinner or less concentrated and may also improve manufacturability. Diluents of the present invention can also serve other functions. For example, a diluent can also serve as a sweetener. Representative diluents include, but are not limited to, sucrose, sorbitol, xylitol, dextrose, fructose, malitol, sugar potassium, aspartame, saccharin, saccharin sodium, and mixtures thereof. A preferred diluent of the present invention is sucrose.

The term “flavoring agent,” as used herein, refers to an agent or a mixture of agents that adds flavor to a mixture. Representative flavoring agents include, but are not limited to, artificial strawberry flavor and artificial cream flavor.

The term “glidant,” as used herein, refers to an agent or a mixture of agents that facilitates the flow of powders in the manufacturing process. Representative glidants include, but are not limited to, colloidal silicon dioxide, talc, fumed silica, magnesium stearate, calcium stearate, magnesium trisilicate, powdered cellulose, starch, tribasic calcium phosphate, and mixtures thereof. A preferred glidant of the present invention is colloidal silicon dioxide.

The term “lubricant,” as used herein refers to an agent or a mixture of agents that lessens or prevents friction. Representative lubricants include, but are not limited to, magnesium stearate, calcium stearate, zinc stearate, magnesium oxide, stearic acid, sodium stearyl fumarate, sodium lauryl stearate, hydrogenated vegetable oil, corn starch, colloidal silicon dioxide, talc, and mixtures thereof. A preferred lubricant of the present invention is magnesium stearate.

The term “preservative,” as used herein, refers to an agent or mixture of agents that is used to protect a composition against antimicrobial (e.g., yeast, mold, bacteria) activity. Representative preservatives include, but are not limited to, sodium benzoate, benzoic acid, ethylenediaminetetraacetic acid, sorbic acid, benzethonium chloride, benzalkonium chloride, bronopol, butyl paraben, methyl paraben, ethylparaben, propyl paraben, thiomerosol, sodium propionate, chlorhexidine, chlorobutanol, chlorocresol, cresol, imidurea, phenol, phenylmercuric salts, potassium sorbate, propylene glycol, and mixtures thereof. A preferred preservative of the present invention is sodium benzoate.

The term “viscosity enhancer,” as used herein, refers to an agent or a mixture of agents that increases the thickness of a liquid thereby making it slow to flow. For example, in a suspension a viscosity enhancer will help to keep the active ingredient suspended to allow accurate dosing. Representative viscosity enhancers include, but are not limited to, xantham gum, guar gum, acacia, povidone, alginic acid, sodium alginate, propylene glycol alginate, carbomer, carboxymethylcellulose calcium, carboxymethylcellulose sodium, ethylcellulose, gelatin, ethylcellulose, hydroxyethyl cellulose, hydroxypropyl cellulose, polydextrose, carrageenan, methylcellulose, sucrose, sorbitol, xylitol, dextrose, fructose, malitol, sugar, sodium alginate, tragacanth, hydroxypropyl methylcellulose, bentonite, a polyvinyl alcohol, cetearyl alcohol, colloidal silicon dioxide, and mixtures thereof. A preferred viscosity enhancer of the present invention is a mixture of xantham gum and guar gum.

Cefdinir can be prepared according to the procedures described in U.S. Pat. Ser. No. 4,935,507, issued Jun. 19, 1990 and U.S. Pat. Ser. No. 4,559,334, issued Dec. 17, 1985, both herein fully incorporated by reference.

Example 1 shows the percentage amounts used in the preparation of an 8% cefdinir oral powder formulation. As mentioned earlier, the current marketed Omnicef® for suspension is a 4% (4.2% actual) cefdinir powder by weight. The 8% formula was bioequivalent to the Omnicef® for oral suspension product.

### EXAMPLE 1

| | Percent Used |
|---|---|
| Ingredient | in 8% Formulation |
| Cefdinir | 8.361 |
| Sucrose, NF Extra Fine Granulated | 89.157 |
| Citric Acid, USP Anhydrous Powder | 0.164 |
| Sodium Citrate, USP Anhydrous Powder | 0.098 |
| Sodium Benzoate, NF | 0.164 |
| Xanthan Gum, NF (Xantural 75) | 0.164 |
| Guar Gum, NF | 0.164 |
| Artificial Cream Flavor 610979U-PFW | 0.131 |
| Colloidal Silicon Dioxide Anhydrous, NF | 0.066 |
| Artificial Strawberry Flavor DY04359 | 0.393 |
| Artificial Strawberry Flavor FD9581-S | 0.787 |
| Magnesium Stearate, NF | 0.351 |

Examples 2 and 3 show percentage amounts that can be used in the preparation of 6% and 10% cefdinir oral powder formulations.

### EXAMPLE 2

| | Percent Used |
|---|---|
| Ingredient | in 6% Formulation |
| Cefdinir | 6.000 |
| Sucrose, NF Extra Fine Granulated | 91.518 |
| Citric Acid, USP Anhydrous Powder | 0.164 |
| Sodium Citrate, USP Anhydrous Powder | 0.098 |
| Sodium Benzoate, NF | 0.164 |
| Xanthan Gum, NF (Xantural 75) | 0.164 |
| Guar Gum, NF | 0.164 |
| Artificial Cream Flavor 610979U-PFW | 0.131 |
| Colloidal Silicon Dioxide Anhydrous, NF | 0.066 |
| Artificial Strawberry Flavor DY04359 | 0.393 |
| Artificial Strawberry Flavor FD9581-S | 0.787 |
| Magnesium Stearate, NF | 0.351 |

### EXAMPLE 3

| | Percent Used |
|---|---|
| Ingredient | in 10% Formulation |
| Cefdinir | 10.000 |
| Sucrose, NF Extra Fine Granulated | 185.04 |
| Citric Acid, USP Anhydrous Powder | 0.328 |
| Sodium Citrate, USP Anhydrous Powder | 0.196 |
| Sodium Benzoate, NF | 0.328 |
| Xanthan Gum, NF (Xantural 75) | 0.328 |
| Guar Gum, NF | 0.328 |
| Artificial Cream Flavor 610979U-PFW | 0.262 |
| Colloidal Silicon Dioxide Anhydrous, NF | 0.130 |
| Artificial Strawberry Flavor DY04359 | 0.790 |
| Artificial Strawberry Flavor FD9581-S | 1.570 |
| Magnesium Stearate, NF | 0.702 |

What is claimed is:

1. A powder for oral suspension comprising greater than 4.2% by weight of cefdinir.

2. A powder for oral suspension comprising about 6% to about 10% by weight of cefdinir.

3. A powder for oral suspension comprising at least 8.4% by weight cefdinir.

4. A powder for oral suspension comprising
(a) at least 8.4% by weight cefdinir;
(b) a diluent; and
(c) a buffering agent.

5. A powder for oral suspension of claim 4 wherein the diluent is selected from the group consisting of sucrose, sorbitol, xylitol, dextrose, fructose, malitol, sugar potassium, aspartame, saccharin, saccharin sodium, and mixtures thereof.

6. A powder for oral suspension of claim 5 wherein the diluent is sucrose.

7. A powder for oral suspension of claim 4 wherein the buffering agent is selected from the group consisting of citric acid, sodium citrate, sodium phosphate, potassium citrate, and mixtures thereof.

8. A powder for oral suspension of claim 7 wherein the buffering agent is a mixture of citric acid and sodium citrate.

9. A powder for oral suspension comprising:
(a) about 8.4% by weight cefdinir;
(b) about 89.2% by weight diluent;
(c) about 0.26% by weight buffering agent;
(d) about 0.16% by weight preservative;
(e) about 0.33% by weight viscosity enhancer;
(f) about 1.31% by weight flavoring agent;
(g) about 0.07% glidant; and
(h) about 0.35% lubricant.

10. A powder for oral suspension of claim 9 wherein the diluent is selected from the group consisting of sucrose, sorbitol, xylitol, dextrose, fructose, malitol, sugar potassium, aspartame, saccharin, saccharin sodium, and mixtures thereof.

11. A powder for oral suspension of claim 10 wherein the diluent is sucrose.

12. A powder for oral suspension of claim 9 wherein the buffering agent is selected from the group consisting of citric acid, sodium citrate, sodium phosphate, potassium citrate, and mixtures thereof.

13. A powder for oral suspension of claim 12 wherein the buffering agent is a mixture of citric acid and sodium citrate.

14. A powder for oral suspension of claim 9 wherein the preservative is selected from the group consisting of sodium benzoate, benzoic acid, ethylenediaminetetraacetic acid, sorbic acid, benzethonium chloride, benzalkonium chloride, bronopol, butyl paraben, methyl paraben, ethylparaben, propyl paraben, thiomerosol, sodium propionate, chlorhexidine, chlorobutanol, chlorocresol, cresol, imidurea, phenol, phenylmercuric salts, potassium sorbate, propylene glycol, and mixtures thereof.

15. A powder for oral suspension of claim 14 wherein the preservative is sodium benzoate.

16. A powder for oral suspension of claim 9 wherein the viscosity enhancing agent is selected from the group consisting of xantham gum, guar gum, acacia, povidone, alginic acid, sodium alginate, propylene glycol alginate, carbomer, carboxymethylcellulose calcium, carboxymethylcellulose sodium, ethylcellulose, gelatin, ethylcellulose, hydroxyethyl cellulose, hydroxypropyl cellulose, polydextrose, carrageenan, methylcellulose, sucrose, sorbitol, xylitol, dextrose, fructose, malitol, sugar, sodium alginate, tragacanth, hydroxypropyl methylcellulose, bentonite, a polyvinyl alcohol, cetearyl alcohol, colloidal silicon dioxide, and mixtures thereof.

17. A powder for oral suspension of claim 16 wherein the viscosity enhancing agent is a mixture of xantham gum and guar gum.

18. A powder for oral suspension of claim 9 wherein the glidant is selected from the group consisting of colloidal silicon dioxide, talc, fumed silica, magnesium stearate, calcium stearate, magnesium trisilicate, powdered cellulose, starch, tribasic calcium phosphate, and mixtures thereof.

19. A powder for oral suspension of claim 18 wherein the glidant is colloidal silicon dioxide.

20. A powder for oral suspension of claim 9 wherein the lubricant is selected from the group consisting of magnesium stearate, calcium stearate, zinc stearate, magnesium oxide, stearic acid, sodium stearyl fumarate, sodium lauryl stearate, hydrogenated vegetable oil, corn starch, colloidal silicon dioxide, talc, and mixtures thereof.

21. A powder for oral suspension of claim 20 wherein the lubricant is magnesium stearate.

22. A powder for oral suspension comprising:
(a) about 8.36% by weight cefdinir;
(b) about 89.16% by weight sucrose;
(c) about 0.16% by weight citric acid;
(d) about 0.10% by weight sodium citrate;
(e) about 0.16% by weight sodium benzoate;
(f) about 0.16% by weight xantham gum;
(g) about 0.16% by weight guar gum;
(h) about 1.31% by weight flavoring agent;
(i) about 0.06% colloidal silicon dioxide; and
(j) about 0.35% magnesium stearate.

23. A method of treating acute bacterial otitis media, pharyngitis and tonsillitis with a oral suspension of cefdinir wherein said suspension is made by reconstituting a powder comprising greater than 4.2% by weight of cefdinir.

24. A method of treating acute bacterial otitis media, pharyngitis and tonsillitis with a oral suspension of cefdinir wherein said suspension is made by reconstituting a powder comprising at least 8.4% cefdinir.
